(19) United States
(12) Patent Application Publication
(10) Pub. No.: US 2023/0363654 A1
(43) Pub. Date: Nov. 16, 2023

(54) BEAMFORMING SYSTEMS AND METHODS FOR DETECTING HEART BEATS

(71) Applicant: University of Washington, Seattle, WA (US)

(72) Inventors: Shyamnath Gollakota, Seattle, WA (US); Anran Wang, Seattle, WA (US); Arun R. Mahankali Sridhar, Seattle, WA (US)

(73) Assignee: University of Washington, Seattle, WA (US)

(21) Appl. No.: 18/246,175

(22) PCT Filed: Sep. 24, 2021
(86) PCT No.: PCT/US2021/052018

§ 371 Date: Mar. 21, 2023

Related U.S. Application Data
(60) Provisional application No. 63/082,960, filed on Sep. 24, 2020.

(51) Int. Cl. A61B 5/024; A61B 5/00

(52) U.S. Cl. CPC A61B 5/02405; A61B 5/6887; A61B 5/7235

(57) ABSTRACT

Examples of systems and methods are described for detecting heart beats of a subject. The systems and methods may be based on motion of the subject due to cardiac activity, and may operate without contact with the subject. Example systems may provide an interrogation signal to the subject. Reflected signals from the subject are incident on a microphone array. The reflected signals may be processed and beamformed using a set of beamforming weights. The beamforming weights may be selected in a manner to reduce components of the reflected signals due to breathing motion of the subject while increasing the relative contribution of the reflected signals due to cardiac activity. The beamformed signal may provide a waveform indicative of heart beats. Inter-beat intervals, heart rates, and/or other health metrics may be calculated based on the waveform.

### CROSS-REFERENCE TO RELATED APPLICATIONS

This application claims the benefit under 35 U.S.C. § 119 of the earlier filing date of U.S. Provisional Application Ser. No. 63/082,960 filed Sep. 24, 2020, the entire contents of which are hereby incorporated by reference in their entirety for any purpose.

### STATEMENT REGARDING RESEARCH & DEVELOPMENT

This invention was made with government support under Grant No. 1812559, awarded by the National Science Foundation. The government has certain rights in the invention.

### TECHNICAL FIELD

Examples described herein relate generally to heart beat detection. Examples of heart beat detection using smart speakers are described.

### BACKGROUND

Heart rhythm assessment is used in diagnosis and management of many cardiac conditions and to study heart rate variability in healthy individuals. Clinical heart rhythm assessment depends on reliable acquisition of beat-to-beat intervals of the heart, also known as the R-R intervals. Physiologically, the R-R interval represents the time between successive ventricular depolarizations of the heart. Acquisition and assessment of R-R interval irregularity is used for diagnosing many cardiac arrhythmias and to study heart rate variability in healthy individuals. Although frequency domain analysis can estimate average heart rate in regular and quasi-periodic heart rhythm conditions, it fails when the rhythm is irregular, which is common in pathological conditions such as atrial fibrillation. R-R intervals are conventionally measured by identifying individual heart beats extracted using electrocardiography (ECG). This approach works for both regular and irregular rhythms but requires physical contact with the skin to operate.

### BRIEF DESCRIPTION OF THE DRAWINGS

FIG. 1 is a schematic illustration of a system arranged in accordance with examples described herein.

FIG. 2 is a schematic illustration of signal processing performed by systems described herein.

FIG. 3A depicts, for a healthy subject, an ECG trace and a corresponding waveform generated in accordance with examples described herein.

FIG. 3B depicts, for a subject experiencing atrial fibrillation, an ECG trace and a corresponding waveform generated in accordance with examples described herein.

### DETAILED DESCRIPTION

Certain details are set forth herein to provide an understanding of described embodiments of technology. However, other examples may be practiced without various of these particular details. In some instances, well-known circuits, control signals, timing protocols, and/or software operations have not been shown in detail in order to avoid unnecessarily obscuring the described embodiments. Other embodiments may be utilized, and other changes may be made, without departing from the spirit or scope of the subject matter presented here.

Examples of systems described herein may be used to acquire individual heart beats using smart speakers in a fully contact-free manner. Methods described herein can be considered to transform the smart speaker into a short-range active sonar system and measure heart rate and inter-beat intervals (e.g., R-R intervals) for both regular and irregular rhythms. In some examples, the smart speaker emits inaudible 18-22 kHz sound and receives echoes reflected from the human body that encode sub-mm displacements due to heart beats.

Smart speaker technology is rapidly evolving and may provide a reliant and convenient platform for the next generation of health monitoring solutions. Indeed, the increasing adoption of smart speakers in hospitals and homes could provide a mechanism to realize the potential for examples of contactless cardiac rhythm monitoring systems described herein.

A non-contact solution for heart rhythm monitoring may offer several advantages. It can monitor infectious and contagious patients where cleaning of contact-based devices can be time consuming and burdensome, monitor patients in home isolation and quarantine settings, and/or benefit patients with skin allergies who are intolerant to wearable and contact-based devices. Contactless rhythm acquisition may also be valuable in the modern telemedicine era, whereby patients' self-administered rhythm analysis are communicated to their physician. The benefits of a self-administered test are numerous, and may include the ability to connect patients living in rural areas to physicians, screening patients for atrial fibrillation remotely, and obtaining clinical trial data without the need for an in-person visit.

The widespread adoption of high quality smart speakers equipped with multiple microphones presents an opportunity for contactless monitoring of human body and internal organ functions. Google Nest smart devices can already determine a user's distance on its smart speaker by emitting soft, inaudible acoustic signals and analyzing their reflections from the human body. Apple HomePod and Amazon Echo devices support an array of six and seven microphones, respectively, that are used for sophisticated acoustic processing.

Examples described herein provide contactless system for monitoring cardiac rhythm using smart speakers that can identify individual heart beats in both regular and irregular rhythms. Examples of methods described herein may extract both heart rate and R-R intervals by generally transforming a smart speaker into a short-range active sonar system. An active sonar based approach to contactless monitoring has the distinct benefit of scalability vis-a-vis smart speakers. Unlike doppler radar and optical vibrocardiography, active sonar hardware components (e.g., multiple microphones and speaker) are ubiquitous in smart speakers. Further, in contrast to approaches that use facial photoplethysmographic signals, which raise privacy issues due to their use of cameras, active sonar can operate using inaudible acoustic signals and does not require the capturing of audible sounds.

Examples of methods and devices described herein generally include the use of a smart speaker to emit interrogation signals (e.g., 18-22 kHz inaudible sound signals) that are reflected off the human body and received by multiple microphones (e.g., a microphone-array). Methods are described to 1) analyze these signals and detect the subtle motion of the chest wall caused by the heart's apical impulse as well as by arterial pulsations on the body's surface, and 2) separate these signals from much larger breathing motions and ambient noise. An example smart speaker implementing these methods that is placed in front of a subject less than a meter away can identify individual heart beats and extract heart rate and R-R intervals for both healthy participants and patients with different cardiac abnormalities. This data could be used for studying heart rhythms, detecting cardiac arrhythmias, and determining heart rate variability.

The ability to monitor cardiac rhythm using smart speakers may raise privacy concerns. The short-range nature of active sonar examples described herein, however, can protect privacy since it uses the direct engagement and implicit consent of the user, who must be within a threshold distance (e.g., a meter) of the speaker and stay relatively still. Frequencies may be used for interrogation signals, e.g., 18-22 kHz acoustic frequencies, that contain little information about audible sounds in the environment. Finally, smart speaker manufacturers generally do not give third party app developers access to raw acoustic signals from individual microphones. Consequently, the smart speaker manufacturers can implement and deploy this capability in a manner that balances the needs and concerns of patients, health care providers and privacy advocates.

A variety of challenges may be presented in endeavoring to measure heart rhythms, including irregular heart rhythms in a contactless system (e.g., a system in which the subject is not contacting an electrode or other contact sensor used to measure heart rhythm). If heart beats were regular, frequency domain analysis may be used to extract the heart motion from the fundamental frequency and its harmonic components. This approach however may not work with irregular heart rhythm since there is no well-defined peak in the frequency domain and the energy is spread across a range of frequencies. Extracting irregular beats may be difficult using acoustic signals since heart beats result in a 0.3-0.8 mm motion on the surface of the human body; this is an order of magnitude smaller than the wavelength of sound at operational frequencies described herein. Further, commodity smart speakers are designed primarily to transmit in the audible frequencies, and the inaudible frequencies they support have a limited bandwidth—4 kHz bandwidth across 18-22 kHz—with a non-ideal frequency response. Unlike ultrasonic devices, commodity smart devices also have a limited sampling rate, about 48 kHz, that produces a low signal-to-noise ratio, making it difficult to achieve the high temporal resolution used to measure the precise timing of each heartbeat. Another complicating factor may be that breathing creates a much larger motion than heart beats on the surface of the body. Though respiration rates are typically lower than heart rates, respiration is not a perfect sinusoidal motion since inhalation and exhalation durations can differ. This creates high frequency components in the breathing motion that interfere with the minute heart beat motion. At low signal-to-noise ratios, this prevents the latter from being reliably separated in the frequency domain using filtering; when the heart signal is weak and overwhelmed by interference from breathing motion, it can become challenging to extract individual heart beats in irregular rhythm.

Examples of systems and methods described herein may address one or more of the challenges described above and/or exhibit one or more of the advantages described above. However, it is to be understood that not all examples of the systems and methods described herein may address all or even any of challenges and/or provide all or even any of the advantages.

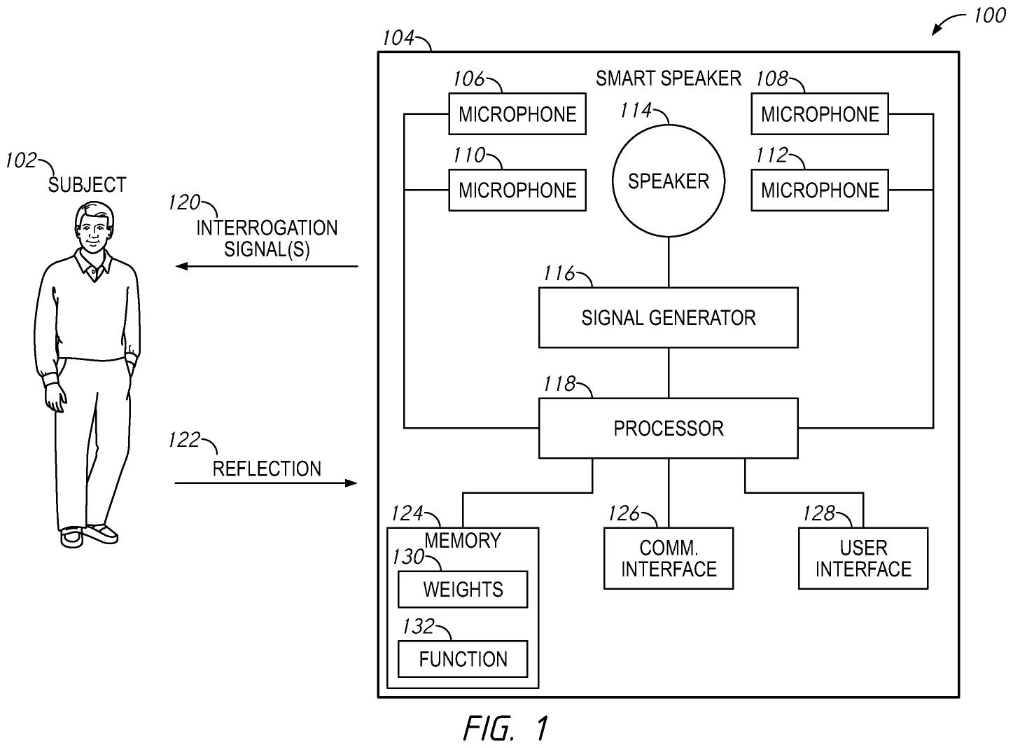

FIG. 1 is a schematic illustration of a system arranged in accordance with examples described herein. The system 100 includes a smart speaker 104 positioned proximate a subject 102. The smart speaker 104 may include microphone 106, microphone 108, microphone 110, and microphone 112. The smart speaker 104 may include speaker 114, signal generator 116, processor 118, comm. interface 126, user interface 128, and memory 124. The memory 124 may store weights 130 and/or function 132. The speaker 114 may be coupled to signal generator 116. The signal generator 116 may be coupled to processor 118. The microphone 106, microphone 110, microphone 108, and microphone 112 may be coupled to processor 118. The processor 118 may be coupled to memory 124, comm. interface 126, and/or user interface 128. During operation, the smart speaker 104 may provide interrogation signal(s) 120 to the subject 102 and receive reflection 122 from the subject 102.

The components shown in FIG. 1 are exemplary only. In other examples, fewer, additional, and/or different components may be used.

Examples of systems described herein may include one or more electronic devices which may provide interrogation signals to a subject and/or receive interrogation signals from a subject, such as smart speaker 104. While smart speaker 104 is shown in FIG. 1, other devices may be used which incorporate the components and/or functions described with reference to smart speaker 104. For example, one or more computers, tablets, smart phones, cellular phones, laptops, and/or wearable devices may be used to implement smart speaker 104. In some examples, the smart speaker 104 may be implemented using a GOOGLE NEST, an APPLE HOMEPOD, and/or an AMAZON ECHO device.

Examples of systems described herein may provide interrogation signals to and/or receive reflected signals from a subject, such as subject 102. Generally, any subject having a heart beat may be used—e.g., one or more humans, adults, children, babies, animals, pets, dogs, and/or cats. While a single subject 102 is shown in FIG. 1, in some examples, multiple subjects may be present. In some examples, electronic devices described herein may discriminate between signals received from each of multiple subjects. The subject may be in generally any position. In some examples, the subject may remain still during interrogation by the interrogation signal. In some examples, the subject may be standing, sitting, driving, and/or sleeping. The subject may generally face the smart speaker 104, although the smart speaker 104 may be placed at an angle from the subject in some examples. During measurement of heart rhythm, in some examples, the subject may generally hold still.

Examples of electronic devices described herein (e.g., smart speaker 104) may include one or more microphones, such as microphone 106, microphone 108, microphone 110, and microphone 112 of FIG. 1. While four microphones are shown in the example of FIG. 1, generally any number may be used, including 2, 3, 4, 5, 6, 7, 8, 9, or 10 microphones. The set of microphones may be referred to as a microphone array. The microphones may be positioned in a grid, such as a rectangular array, or may be positioned in other arrangements. In some examples, the microphones may be positioned in a circular array. The microphones may be positioned such that, during operation, they receive reflected signals from a subject. The different position of each microphone relative to the subject 102 allows a different portion of the reflected signals to be incident on each of the microphones. Note that a distance resolution achievable by systems described herein may depend on various factors that affect phase error—e.g., hardware components, circuit design and interference control, operating system and driver to support high-throughput audio signals, and the techniques themselves.

Examples of electronic devices described herein (e.g., smart speaker 104) may include a speaker, such as speaker 114. Although a single speaker is shown in FIG. 1, it is to be understood that any number of speakers may be used. In some examples, a speaker may have parameters selected based on intended operations herein. For example, a speaker may have a response which is optimized and/or better at certain target frequencies (e.g., frequencies of the interrogation signal, such as 18-22 kHz). In some examples, a speaker may include and/or be coupled to a directional tweeter which may rotate toward and direct the interrogation signal toward the subject. In some examples, sampling rates and bit resolutions of the speaker may be selected for particular performance levels. In some examples, the speaker 114 may have a sampling rate of 48 kHz.

Examples of electronic devices described herein (e.g., smart speaker 104) may include a signal generator, such as signal generator 116. Example signal generators may include one or more modulators, amplifiers, and/or other circuitry to generate a signal. The signal generated by the signal generator may be used to drive a speaker, such as speaker 114. During operation, the signal generator 116 may drive the 114 to produce the interrogation signal(s) 120.

Accordingly, examples of electronic devices described herein, such as smart speaker 104, may be used to generate interrogation signals, such as interrogation signal(s) 120. Examples of interrogation signals include frequency modulated continuous wave (FMCW) signals, white noise, continuous tone signals, and/or other modulated signals. In some examples, the interrogation signal(s) 120 may be between 18 and 22 kHz (e.g., FMCW signals between 18 and 22 kHz). In some examples, the interrogation signal(s) 120 may be between 18 and 30 kHz (e.g., FMCW signals between 18 and 30 kHz). In some examples, the interrogation signal(s) 120 may be between 25 and 30 kHz (e.g., FMCW signals between 25 and 30 kHz). In some examples, the interrogation signal(s) 120 may be between 22 and 25 kHz (e.g., FMCW signals between 22 and 25 kHz). In some examples, the interrogation signal(s) 120 may be between 20 and 36 kHz (e.g., FMCW signals between 20 and 36 kHz). Frequencies higher than 30 kHz may be used in some examples. In some examples, the interrogation signal(s) 120 may have one or more acoustic frequencies. In some examples, the interrogation signal(s) 120 may be inaudible signals (e.g., signals above a frequency generally audible to humans). In this manner, the interrogation signals provided by the smart speaker 104 may generally not be heard by humans.

Examples of electronic devices described herein may include one or more processors, such as processor 118 of FIG. 1. The processor 118 may be implemented using one or more processors (e.g., central processing unit (CPU) and/or graphics processing unit (GPU)) and/or processing circuitry, such as one or more digital signal processors, application specific integrated circuits (ASICs), field programmable gate arrays (FPGAs), controllers, and/or microcontrollers. The processor 118 may be coupled to each of the microphones, e.g., microphone 106, microphone 108, microphone 110, and microphone 112 of FIG. 1. The processor 118 may accordingly be provided with signals corresponding to the reflection 122 from the subject 102. The processor 118 may be used in conjunction with software and/or firmware (e.g., executable instructions stored on a memory such as memory 124) to analyze reflected signals provided by the microphones to determine (e.g., generate) a waveform indicative of heart beats of a subject (e.g., subject 102).

Examples of processors described herein, such as processor 118 may be used to beamform multiple received signals (e.g., signals provided by microphone 106, microphone 108, microphone 110, and/or microphone 112) generated responsive to signals incident on the microphones. Beamforming generally refers to combining (e.g., adding and/or subtracting) the signals in a weighted manner. A processor may access and/or utilize one or more beamforming weights to conduct beamforming—e.g., a weight may be associated with each of the microphones.

Examples of electronic devices described herein, such as smart speaker 104 of FIG. 1 may include memory, such as memory 124. The memory may be implemented using any variety of memory or combinations of memories (e.g., random access memory (RAM), read only memory (ROM), solid state drive(s), SD cards, Flash). The memory may be used to store beamforming weights, such as weights 130 of FIG. 1. In some examples, the beamforming weights may be complex weights (e.g., having a real and imaginary portion). To beamform the reflected signals provided to the processor 118, the processor 118 may weight the reflected signals using the beamforming weights (e.g., weights 130), and combine (e.g., sum and/or subtract) the weighted signals.

Examples of processors described herein may accordingly beamform multiple received signals to generate a waveform that is indicative of heart beats of a subject (e.g., heart beats of subject 102). The beamformed signal provided by the processor may be indicative of heart beats of the subject, because the beamforming weights may in some examples be selected such that a weighted combination of received signals using those beamforming weights may be indicative of heart beats of the subject.

Examples of processors described herein, such as processor 118 of FIG. 1, may be used to calculate beamforming weights. For example, the processor 118 may be used to calculate weights 130. The beamforming weights may be calculated in such a manner that the use of the beamforming weights to beamform received signals may result in a waveform indicative of heart beats of a subject. In some examples, the beamforming weights may be associated with a particular interrogation signal and/or set of signals.

The beamforming weights may be calculated by evaluating a function. Examples of memory described herein, such as memory 124 may be used to store the function, e.g., function 132. The function 132 may be implemented using, for example, data representing a function. In some examples, the function may be evaluated using the processor 118 multiple times (e.g., using different weight candidates), and the beamforming weights may be calculated by selecting the weight candidates which satisfied a particular constraint on the function (e.g., maximized, minimized, optimized). In some examples, evaluating the function may include performing (e.g., using the processor 118) one or more machine learning techniques, such as one or more unsupervised learning techniques (e.g., gradient ascent technique), and/or using one or more neural networks. In some examples, the function may be used to increase a portion of the resulting beamformed signal (e.g., waveform) corresponding to the heart beats and to decrease a portion of the resulting beamformed signal (e.g., waveform) corresponding to breathing motion of the subject. Accordingly, the function may be based in part on expected frequencies of respiration and heart beats.

Examples of processors described herein, such as processor 118, may be used to calculate a health metric based on the beamformed reflected signals (e.g., waveform). The health metric may be, for example, a heart rate, heart rate variability, inter-beat interval, and/or combinations thereof. The processor 118 may be used to segment the waveform to identify segments of the waveform corresponding to each heartbeat. Based on a number of segments per time and/or a length of the segments, the processor 118 may calculate an inter-beat interval and/or heart rate. Differences in the inter-beat interval may be used by the processor 118 to calculate heart rate variability.

Examples of electronic devices described herein may include one or more communication interfaces, such as comm. interface 126 of FIG. 1. The communication interface may be coupled to a processor and may be used to receive and/or transmit data. For example, the comm. interface 126 may be used to transmit a waveform indicative of the heart beats of subject 102 to another computing device. The comm. interface 126 may be implemented using a wired and/or wireless interface including, but not limited to, WiFi, BLUETOOTH, cellular, or combinations thereof.

Examples of electronic devices described herein may include one or more user interfaces, such as user interface 128 of FIG. 1. The user interface may be implemented, for example, using one or more display(s), keyboards, mice, touchscreens, buttons, lights, vibrators, or combinations thereof. In some examples, a user (e.g., a person and/or a software process) may provide, through the user interface 128, an indication to begin providing interrogation signal(s) 120. Responsive to the indication, the signal generator 116 may drive the speaker 114 to produce the interrogation signal(s) 120. In some examples, the user interface 128 may display a waveform based on the received signals at the microphones that is indicative of heart beats of a subject. In some examples, the user interface 128 may display health metric(s) about the subject 102 calculated using the beamformed waveform generated by the processor 118.

During operation, electronic devices described herein may be utilized to determine heart beats and/or related health metrics of a subject. The determination may be made without contact between the electronic device and the subject in some examples.

For example, the smart speaker 104 may provide interrogation signal(s) 120 to the subject 102. The signal generator 116 may drive the speaker 114 to generate the interrogation signal(s) 120. The interrogation signal(s) 120 may reflect off the subject 102, providing at least reflection 122. Other reflections may additionally be generated. Movement of the subject 102, including movement due to heart beats of the subject 102 may change the reflection 122.

The reflection 122 and/or additional reflections of the interrogation signal(s) 120 may be incident on one or more microphones of an electronic device—such as microphone 106, microphone 108, microphone 110, and microphone 112 of FIG. 1. The microphones may transduce the reflection and may provide reflected signals to processor 118. In some examples, the processor 118 may calculate beamforming weights for use in beamforming the received signals. For example, the processor 118 may calculate weights 130 at least in part by using the reflected signals to evaluate function 132. Candidates weights may be used in the function 132, and the beamforming weights may be selected candidate weights that meet a constraint on the function (e.g., maximize, minimize, etc.). In some examples, a portion of the reflected signals may be used by the processor 118 to calculate the weights 130. Then, subsequent portions of the reflected signals (e.g., received at times after the calculation of the weights) may be beamformed to generate a waveform indicative of heart beats. In other examples, the weights may be predetermined and present in memory 124 and used by the processor 118 to generate a waveform indicative or heart beats.

The processor 118 (or another computing device and/or processor) may utilize the waveform indicative of heart beats to calculate one or more health metrics including inter-beat interval and/or heart rate. The processor 118 (or another computing device and/or processor) may utilize the health metrics to detect a health condition based on the health metric(s)—e.g., atrial fibrillation, flutter, congestive heart failure, arrhythmia, or combinations thereof.

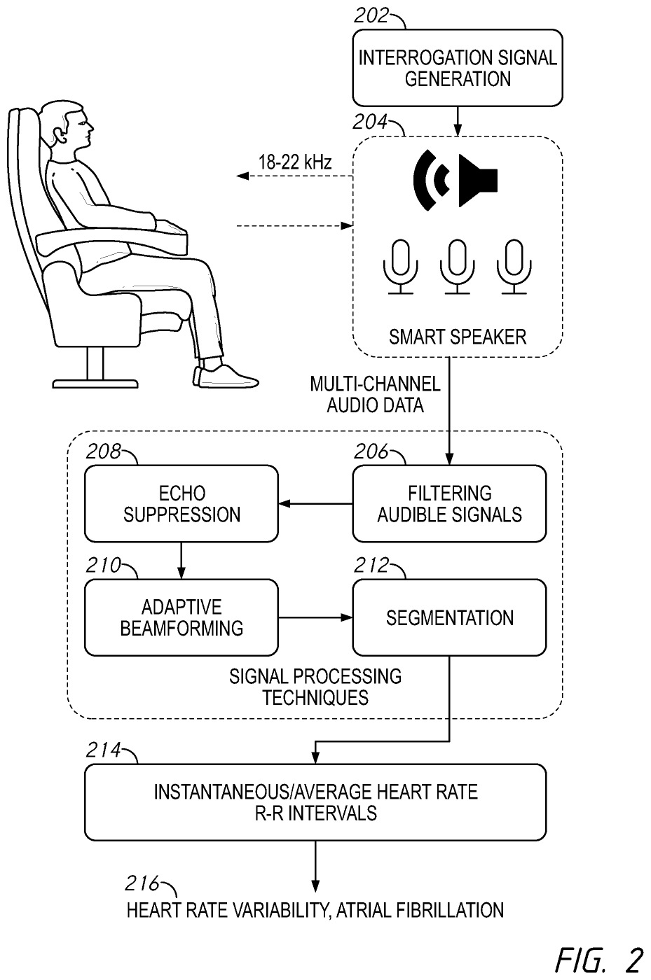

FIG. 2 is a schematic illustration of signal processing performed by systems described herein. The signal processing workflow shown in FIG. 2 includes signal generation 202, transduce 204, filtering 206, echo suppression 208, beamforming 210, segmentation 212, health metric calculation 214, and disease detection 216. The signal processing operations shown in FIG. 2 may be performed by electronic devices described herein, including smart speaker 104 of FIG. 1, for example. The operations shown in FIG. 2 are exemplary. The operations shown in FIG. 2 may be performed in circuitry and/or using one or more processors, such as processor 118 of FIG. 1 in accordance with software and/or firmware. Additional, fewer, and/or different operations may be used in other examples. The operations may be arranged in other orders in other examples.

In signal generation 202, interrogation signal(s) may be generated. For example, the signal generator 116 of FIG. 1 may be used to generate an interrogation signal, such as an FMCW signal, in some examples having a linearly increasing frequency in an inaudible frequency range, such as 18-24 kHz. Generally, an interrogation signal may be used which has good spectral efficiency. In some examples, white noise may be used. In some examples, FMCW signals may be used. Example FMCW signals include chirp blocks. Each chirp block occurs over a time period, and has a linearly changing amplitude between an initial frequency and a final frequency. For example, the time period of a chirp may be 50 ms in some examples, although other periods may be used including, but not limited to 10 ms, 20 ms, 30 ms, 40 ms, 60 ms, 70 ms, 80 ms, 90 ms, 100 ms. Initial and final frequencies may be selected to be outside an audible range in some examples. An example initial frequency may be 18 kHz. An example final frequency may be 24 kHz in some examples, 30 kHz in other examples. Other starting and/or stopping frequencies may be used in other examples.

Mathematically, an FMCW signal is given by:

$$x ( t ) = cos ( 2 π {f}_{0} t + π \frac{F}{T} {t}^{2} ) , t ϵ [ 0 , T ) \qquad ( Equation 1 )$$

where f₀ is the initial frequency, t is time, F is the frequency difference between the initial and the final frequency—such that the final frequency=f₀+F, and T is the period of the chirp block.

A discrete Fourier transform (DFT) may be performed (e.g., using processor 118 of FIG. 2 and/or one or more transform blocks) to obtain a frequency domain representation of the chirp block (e.g., a frequency domain representation of the interrogation signal). The phase of the interrogation signal may be computed (e.g., using processor 118 of FIG. 1) as Ø_{FMCW}(f).

In transduce 204, a speaker is used to transmit the interrogation signal to a subject, and one or more microphones are used to receive reflection(s) of the interrogation signal from the subject. For example, the speaker 114 of FIG. 1 may be driven by the signal generator 116 to provide an interrogation signal to a subject. In some examples, the signal generator may repeat one or more chirp blocks of an FMCW signal. The microphone 106, microphone 108, microphone 110, and microphone 112 may be used to receive the reflected signals. The reflected signals may be transduced by the microphones to electronic reflected signals. The reflected signals provided by the microphones may be multi-channel audio signals—e.g., multi-channel audio data. The reflected signals provided by the microphones may be coupled to a processor, such as processor 118 of FIG. 1, for further signal processing.

Continuing on in FIG. 2, the reflected signals (e.g., audio data representative of the reflected signals) may be processed using any number of signal processing techniques. For example, the processor 118 of FIG. 1 may operate on the reflected signals in accordance with software, firmware, or combinations thereof. For example, the memory 124 may be encoded with executable instructions for performing one or more of the signal processing techniques described herein. In some examples, circuitry may be used to perform one or more of the signal processing techniques described herein.

In filtering 206, the reflected signals may have audible signals filtered out. For example, sounds of the subject or others speaking, coughing, or making other audible sounds may be filtered out. Other ambient audible sounds may be filtered out in filtering 206—such as ambient noises, pet noises, traffic or other noises. In this manner, background noise may be filtered out. The processor 118 of FIG. 1 may perform the filtering and/or one or more filters may be provided to perform the filtering. At an output of the filter, the remaining data may not contain information regarding speech or other potentially private activities occurring in the vicinity of the subject.

In echo suppression 208, reflected signals may be eliminated which may arrive from distances greater than a threshold distance. For example, the threshold distance may be 1 meter in some examples. Other threshold distances include, but are not limited to, 2 meters, 0.5 meters, or 3 meters. Other threshold distances may be used in other examples. A reason it may be advantageous to filter out reflected signals from larger distances is that cardiac motion of the subject may be considered to be relatively minute. The reflected signal due to this relatively small motion of the subject due to cardiac activity can risk being drowned out by reflections corresponding to coarse motion from distant locations. Removing reflections received from distance locations may allow example systems and methods described herein to more accurately analyze reflected signals due to smaller cardiac motion.

In order to filter reflections received from a threshold distance, the impulse response of the acoustic channel may be extracted. For example, the processor 118 of FIG. 1 may calculate an impulse response of the channel over which a reflection was received. The impulse response generally represents the times-of-arrival of the various reflections from the speaker to the microphone. Those that took longer times to arrive are those that were received from longer distances.

In some examples, to compute the impulse response of the acoustic channel on each microphone, transforms (e.g., DFTs) may be performed over signal blocks of duration T (e.g., the duration of an FMCW chirp in some examples). DFTs may be performed with a sliding window, ΔT. In one example, the duration T=50 ms and ΔT=10 ms. In such an example, this provides an effective sampling rate of 100 Hz for the output cardiac signal. Other sliding window durations may be used in other examples. Consider the i^{th} block on the j^{th} microphone as y^{(i,j)}(t). Performing a DFT over this signal gives

$${Y}^{( i , j )} ( f ) = {\sum }_{t = 0}^{T} {y}^{( i , j )} ( t ) {e}^{- \frac{j 2 π ft}{T}} \qquad ( Equation 2 )$$

Equalization may then be performed (e.g., by processor 118 of FIG. 1) to transform the received FMCW chirp (e.g., in the reflected signals) into an impulse response. To do this, the phase of the FMCW chirp, Ø(f), is cancelled in the frequency domain. Since the sliding window resulted in a timing synchronization offset, iΔT mod T, in the FMCW signal, it introduced an additional phase offset in the frequency domain,

$$- 2 π f \frac{Δ T}{T} i .$$

Frequency domain equalization may be performed to cancel both these phases to obtain,

$${Ψ}^{( i , j )} ( f ) = {e}^{- j ϕ ( f ) + j 2 π f \frac{Δ T}{T} i} {Y}^{( i , J )} ( f ) \qquad ( Equation 3 )$$

The time-domain impulse response of the acoustic channel was then obtained by performing an inverse DFT to obtain:

$${ψ}^{( i , j )} ( t ) = {\sum }_{f = {f}_{0} T}^{( {f}_{o} + F ) T} {e}^{\frac{j 2 π ft}{T}} {Ψ}^{( i , j )} ( f ) \qquad ( Equation 4 )$$

This impulse response represents the time-of-arrival of the various reflections from the speaker to the microphone.

Since motion of a subject due to cardiac activity is relatively minute, it can be smaller than reflections corresponding to coarse motion from distant locations. Therefore, echo suppression may be performed to eliminate and/or reduce the reflections arriving from the farther distances. The impulse response at time t represents the total energy of the reflections that arrive at time t. To reduce the effect of reflections from distant motion, the impulse responses may be cancelled out and/or reduced at farther distances. The operational range is the distance between the speaker and the subject, D=1 m in some examples, the round-trip time-of-arrival corresponding to this distance is T_{d}=2D/c, where c is the speed of sound. Zeroing the signal after T_{d} in the impulse responses can lead to abrupt changes in the time domain and spectrum leakage in the frequency domain. Instead, ψ^{(i,j)}(t) may be point-wise multiplied with a raised-cosine window W(t) starting at time 0, with a roll-off factor of 1 and length T_{d}. This yields the impulse response after multipath suppression,

\hat{ψ}^{(i,j)}(t)=ψ^{(i,j)}(t)W(t−T_{d}/2) (Equation 5)

A DFT may be performed on this impulse response to obtain \hat{ψ}^{(i,j)}(f), which represents the reflected signals with suppression of distant reflections.

In an analogous manner, in echo suppression 208, signals due to motion of other subjects in a scene may be removed and/or identified. For example, signals having similar channel impulse responses (e.g., time of arrival) may be considered to be arriving from a same subject. Signals with similar channel impulse responses may accordingly in some examples be processed (e.g., through subsequent adaptive beamforming) to generate waveforms on a per-subject basis. In some examples, multiple subject may be supported by using breathing motion to track the location of each participant, and separating the cardiac signals received from different distances. For example, the processor 118 of FIG. 1 may utilized received signals to identify breathing motion, and therefore identify distances of multiple subjects from the smart speaker 104. The processor 118 may separately beamform sets of received signals arrived from those distances corresponding to the multiple subjects to create separate beamformed waveforms indicative of heart beats for each subject.

While filtering and echo suppression are shown and described herein and with reference to FIG. 2, it is to be understood that the various possible filtering and echo suppression operations may be optional in some examples, and may be present in various combinations.

In beamforming 210, the reflected signals from multiple microphones, which may be echo suppressed reflected signals, may be combined in accordance with beamforming weights to form a waveform. For example, the processor 118 of FIG. 1 may utilize weights 130 to beamform the reflected signals. The weights may be calculated such that the waveform generated using the weighted combination of reflected signals is indicative of heart beats of the subject.

Generally, during beamforming 210, the heart rhythm present in the received signals may be separated from breathing motion. Heart rhythm can be irregular, and breathing motion may not be a perfect sinusoidal signal. Therefore, filtering alone may not be effective. Beamforming 210 generally maximizes the signal-to-interference and noise ratio (SINR) by aligning heart beat signals across microphones and frequencies while minimizing the interference from breathing motion and noise. The beamformer (e.g., using processor 118 of FIG. 1) uses complex weights (e.g., weights 130) to combine the signals from different microphones across frequencies. To compute the weights, a function may be used (e.g., function 132), which may be an optimization function. The function may be solved using a gradient ascent algorithm or other learning algorithm. Since there is no assumption of periodic structure to the heart rhythm, the learning algorithm can erroneously detect high-frequency, impulse-like signals caused by abrupt breaths or interference in the environment. Regularization parameters in the function may be used to penalize such abrupt changes.

To help understand why an adaptive beamformer is used, a discussion is provided on how reflected signals due to breathing motion may interfere with reflected signals due to the heart motion. The received acoustic signal at each microphone is a superposition of reflections from various reflectors on the subject, including the chest, abdomen and neck as well as reflections from static objects and noise. Assuming that breathing and heartbeats result in a displacement of approximately 0.5 cm and 0.5 mm, respectively, this results in a phase change of around 3.3 and a 0.3 radian in the acoustic signal. Thus, the received acoustic signal in the complex domain (e.g., the received signals from the microphones of electronic devices described herein, either before or after echo suppression) can be represented as a linear combination of complex numbers corresponding to two arcs, the respiration arc, and the heartbeat arc, in addition to a constant complex offset from static reflections and noise.

The complex numbers corresponding to the respiration arc generally have a repeating motion along the arc, with a quasi-static respiration frequency (Rᵣₑₛₚ) of less than 20 cycles per minute (CPM) in adult humans. Other frequencies may be used for other types of subjects. Projecting an ideal breathing signal onto the real and imaginary components results in sinusoidal waves. However, the breathing motion is not perfectly sinusoidal in practice. As a result, while the majority of breathing energy in the frequency domain is at Rᵣₑₛₚ and its second harmonic (<40 CPM), a non-negligible portion of energy may leak into the higher frequencies that correspond to heart motion.

A heartbeat arc in comparison may be much smaller, and the moving trajectory along each heartbeat arc can thus be approximated as a linear segment. Hence, the projection of the motion along the arc onto the real or imaginary axis is approximately linear to the motion itself. Human heartbeat motion has a mean frequency (Rₕₑₐᵣₜ) between 60-150 CPM. Other frequencies may be used in other kinds of subjects. However, the instantaneous heart rate, which is the reciprocal of the R-R interval, is not necessarily quasi-static.

Without loss of generality, the motion along the heartbeat arc may be modeled as a carrier wave at a frequency Rₕₑₐᵣₜ) that is frequency modulated (FM) with a finite random signal s(t) that changes the beat-to-beat interval. Since heart beats have an average frequency of Rₕₑₐᵣₜ), the modulating signal s(t) has a maximum bandwidth of B=Rₕₑₐᵣₜ/2. The FM modulation signal can then be written as,

$$F M ( t ) = cos ( 2 π {R}_{h e a r t} t + δf {\int }_{0}^{t} s ( τ ) d τ ) \qquad ( Equation 6 )$$

Here Δf is FM frequency deviation. Variations in beat-to-beat intervals in some examples may be assumed to have a maximum frequency such that Δf<Rₕₑₐᵣₜ/2. As a result, the modulated signal has a low modulation index as

$$\frac{Δ f}{B} < 1$$

and may be a narrow-band FM signal. Given Carson's rule, the spectrum of narrow-band FM signals has only one main lobe, and the majority of the energy of the FM signal falls inside Rₕₑₐᵣₜ±B. Further, the spectrum has a long tail that is spread into frequencies outside this range.

In segmentation 212 the waveform indicative of heart beats of the subject may be segmented into segments, each of which represents a heartbeat of the subject. For example, the processor 118 of FIG. 1 may be used to segment the waveform. In other examples, other computing systems and/or processors may receive the waveform and may conduct segmentation.

The analysis demonstrates two main properties of breathing and heart motion signals. First, a non-negligible minority of the energy corresponding to breathing and heart motion can leak between these frequency ranges. Since the respiration motion is much larger than heartbeat motion, it introduces noise in the 60 to 150 cycles per minute frequencies in some subjects and can hide the heartbeat signal. As a result, band-pass filtering may not help to extract heart rhythm from the active sonar signal. Instead, beamforming may be used. Second, most of the energy corresponding to breathing and heart motion falls in non-overlapping frequencies of [0, 40] and [60,150] CPM, respectively, in adult human subjects.

Both properties may be leveraged in the design of a beamforming technique (e.g., a beamformer) for systems described herein. The beamformer may be described as a maximum signal-to-interference and noise ratio (SINR) beamformer. Taking 30 seconds of blocks (e.g., received signals) as training sequences, the beamformer (e.g., implemented by the processor 118 of FIG. 1 in some examples) may combine the received signals across different microphones and frequencies in the impulse response to maximize the heart signal while minimizing the breathing signal and/or noise. The frequency domain impulse response computed over the i^{th} block and j^{th} microphone can be written as,

\hat{Ψ}^{(i,j)}(f)=a_{j,f}Sᵢ^{(resp)}+β_{j,f}Sᵢ^{(heart)}+C_{j,f}+N_{i,j,f} (Equation 7)

Here Sᵢ^{(resp)} and Sᵢ^{(heart)} correspond to the and heart motion signal a, and β are the corresponding weights, C_{j,f} corresponds to the reflections from the static objects in the environment, and N is the noise. At a high level, the optimization problem (e.g., function as described herein) aims to find the matrix H=[h_{j,f}] such that

$$\frac{{Σ}_{i} \vert ( H \cdot β ) {S}_{i}^{( heart )} {\vert}^{2}}{{Σ}_{i} \vert ( H \cdot α ) {S}_{i}^{( resp )} {\vert}^{2} + Var ( H \cdot N )}$$

is maximized (e.g., the contribution of the signal due to cardiac function is generally increased or maximized while the contribution due to respiratory motion is comparatively decreased or minimized), where A·B=Σ_{i,j}A_{i,j}B_{i,j} and Var(·) denotes the variance. H may represent an array or matrix of beamforming weights as described herein. The beamforming weights may be selected to maximize the afore-mentioned function which may be, wholly or partially used to implement the function 132 of FIG. 1 for example. Other functions may be used, including functions which may seek to increase a contribution of the signal due to cardiac function while decreasing that due to respiratory motion.

The structure of respiration and heart signals may be unknown since it varies across people and time. From the preceding analysis, the majority of the energy corresponding to breathing and heart motion lie in non-overlapping frequencies. So, the energy in these frequency ranges may be used as a proxy for breathing and heart motion in the above optimization. Specifically, S(i)=H·\hat{Ψ}^{(i,j)}(f). Three FIR filters may be used: a low-pass filter Wᵣₑₛₚ with a cut-off frequency at an expected highest breathing frequency (e.g., 50 CPM), a band-pass filter Wₕₑₐᵣₜ with a pass-band from a lowest expected heart beat frequency to a highest expected heart beat frequency (e.g., 60-150 CPM in adult humans), and a high-pass filter Wₙₒᵢₛₑ with a cut-off frequency at the highest expected heart beat frequency (e.g., 150 CPM). The filtered signals may be computed (e.g., using processor 118 of FIG. 1) as,

Ŝᵣₑₛₚ=Wᵣₑₛₚ*S,Ŝₕₑₐᵣₜ=Wₕₑₐᵣₜ*S,Ŝₙₒᵢₛₑ=Wₙₒᵢₛₑ*S (Equation 8)

Where * is the convolution operation. In this manner, the three filtered signals may be identified as primarily containing respiratory motion, cardiac motion, or noise. Each filtered signal represents a component of the overall signal, S, due to respiration, heart motion, and noise, respectively.

An objective function may be evaluated using a learning technique to meet a particular criteria. For example, gradient ascent may be used to maximize the following objective function:

[symbol]

(h)=log(∥ [symbol] (Ŝₕₑₐᵣₜ)∥₂²+∥ [symbol] (Ŝₕₑₐᵣₜ)∥₂²+k [symbol] (Ŝₕₑₐᵣₜ)· [symbol] (Ŝₕₑₐᵣₜ))−log(ŜᵣₑₛₚŜ*ᵣₑₛₚ+ŜₙₒᵢₛₑŜ*ₙₒᵢₛₑ) (Equation 9)

Here, ∥A∥₂ is the 2-norm function of vector A, [symbol] (·) and [symbol] (·) represent the real and imaginary part of a complex number, and S* denotes the conjugate of S. A hyper-parameter k is used to constrained the level of coherence of the real (in-phase) and imaginary (quadrature) parts of the heart signal, because they were both linear projections of the same heart motion and hence should have a large correlation. Note that although a band-pass filter is used in this example, it may not be used directly for signal extraction but only as a metric for approximating the SINR. The above objective function may be used to wholly and/or partially implement the function 132 of FIG. 1, for example. After computing H using gradient ascent or other learning technique (e.g., unsupervised learning), the heart rhythm signal Ŝₕₑₐᵣₜ may be extracted. For example, Equation 9 may generally be a function of the beamforming weights, h, since the equation is dependent on the various Ŝ signals which may be dependent on the beamformed signal S as described in Equation 8. Accordingly, beamforming weights H may be identified which may maximize the function shown in Equation 9. The heart rhythm signal may be the waveform indicative of heart beats as described herein.

To avoid local maximum, two techniques may be used during optimization. When random noise in any frequency-microphone pair has dominant energy within the heart rate range, it may be wrongly amplified while maximizing the objective function. Unlike random noise, heartbeat motion should exist in a majority of frequency-microphones pairs. Hence, during the backward process in each iteration of gradient ascent, the weight may be probabilistically chosen to update with a probability, e.g., p=0.6, leaving the other weights unmodified.

The gradient ascent algorithm may also incorrectly converge to a local maximum in some examples that appears to be an impulse-like signal, which can be caused by a subject's abrupt motion. The length of the heartbeat arc, however, should not change abruptly over time because the skin displacement from each heartbeat is proportional to the blood pressure or apical impulse. Thus, the resulting signal should have a stable envelope. To enforce this, a regularization penalty term may be used in the objective function (e.g., in function 132) that is the maximum of the heart signal, e.g., |Ŝₕₑₐᵣₜ|. Thus, the objective function used in a gradient ascent algorithm including the regularization penalty is given by:

[symbol]

(H)=−log (∥ [symbol] (Ŝₕₑₐᵣₜ)∥₂²+∥ [symbol] (Ŝₕₑₐᵣₜ)+∥₂²+kΣ| [symbol] (Ŝₕₑₐᵣₜ) [symbol] (Ŝₕₑₐᵣₜ)|) log (ŜᵣₑₛₚŜ*ᵣₑₛₚ+ŜₙₒᵢₛₑŜ*ₙₒᵢₛₑ+γmax(ŜₕₑₐᵣₜŜ*ₕₑₐᵣₜ)) (Equation 10)

Equation 10 may in some examples be used to wholly and/or partially implement the function 132 of FIG. 1, for example). For example, Equation 10 may generally be a function of the beamforming weights, h, since the equation is dependent on the various Ŝ signals which may be dependent on the beamformed signal S as described in Equation 8. Accordingly, beamforming weights H may be identified which may maximize the function shown in Equation 10. The gradient ascent algorithm may be implemented in software, e.g., using PyTorch, with the parameters k=2, y=0.2 (other parameters may be used in other examples). The step size may be set to an initial value (e.g., 1), and the step size may be reduced (e.g., halved) if the objective function value did not increase every threshold number (e.g., 100) of iterations. Convergence may be met when the step size falls below a threshold (e.g., 0.05). In one example, the gradient ascent technique took an average of 2000 iterations to converge. The optimization may be performed over a first portion of the received signals at the microphones (e.g., the first 30 seconds in some examples) to compute the beamforming matrix, H. The beamforming matrix includes the beamforming weights, which may be used to implement weights 130 of FIG. 1. The weights may be used to combine the remaining portion of the received signals (e.g., the echo suppressed received signals) received after the first portion, and generate a waveform indicative of heart beats.

Note that the technique to calculate the beamforming weights may not use supervised learning in that it may not use ground truth data. Instead, a self-supervised learning technique may be used to determine the beamforming weights. The optimization is self-supervised, e.g., that the inference for one subject does not use ground truth training data for the person or pre-trained model on other people. The self-supervised model may extract the hidden information (e.g., the R-R intervals) by optimizing the above objective function in Equation 9 and/or 10. A reason self-supervision may be advantageous is that different body shapes, positions and the surrounding environments may make a supervised model difficult to generalize. Instead, the beamforming weights may be identified that maximize the signal strength of the heart rhythm motion by solving the optimization problem, without the need for any ground truth training data in some examples.

After the beamforming process converged and the beamforming weights (e.g., H) are obtained, the waveform indicative of heart beats, Sₕₑₐᵣₜ, may be obtained by applying a high-pass filter, e.g., above 50 CPM, or other heart frequency threshold, to the real and imaginary parts of the resulting beamformed signal, S. A high-pass filter may be used instead of a band-pass filter to preserve the high-frequency information and improve temporal resolution in the heart beat signal.

Since beamforming 210 may be imperfect, the waveform output from beamforming 210 may include non-negligible residual interference from respiration motion, which may shift the heart signal back and forth between the in-phase and quadrature phase components of the acoustic signal. The segmentation operation may simultaneously identify the segmenting points and the shift in each segment. Systems described herein may accomplish that by 1) comparing adjacent segments to account for different segment lengths due to irregular R-R intervals and, 2) tracking the shift between in-phase and quadrature-phase components caused by residual breathing motion. The segmentation operation may accordingly combine data from both the in-phase and quadrature phase components of the beamformed waveform.

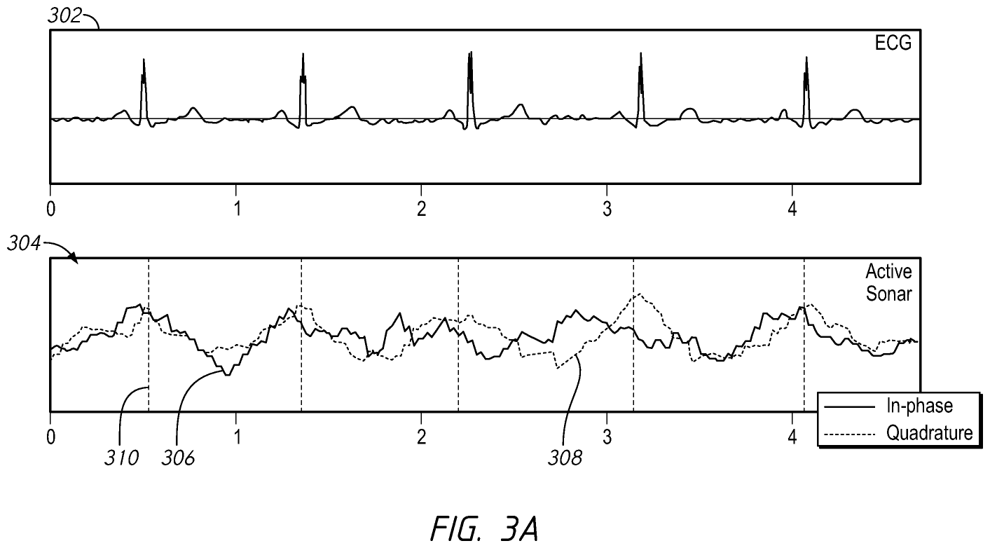

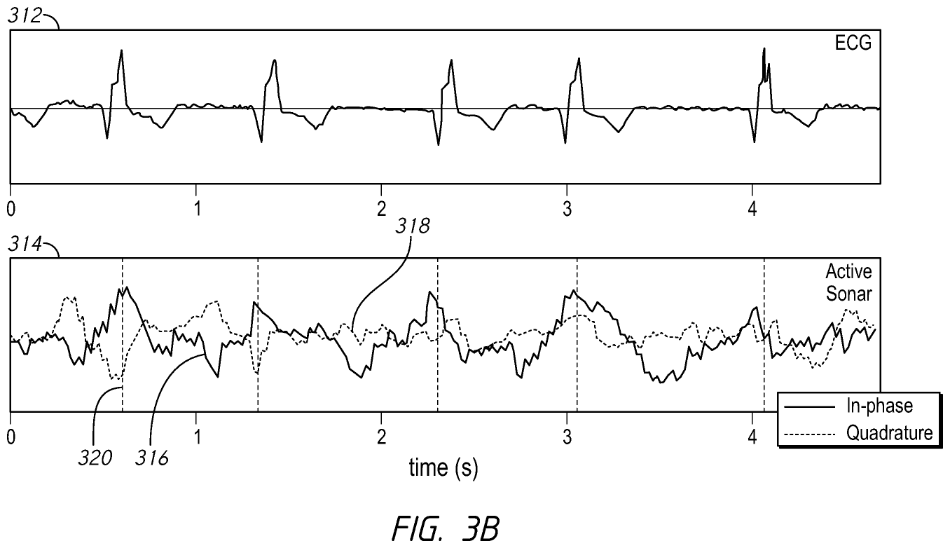

The complex signal, Sₕₑₐᵣₜ, may be segmented into portions corresponding to individual heart beats. Recall imperfect beamforming may leave residual interference from respiratory motion that modulates the heart signal. This may introduce a rotation to the waveform indicative of heart beats, which may change the projection ratio between the real and imaginary components. Thus, the heartbeats may not be observed only on the real (in-phase) or imaginary (quadrature) components (see FIG. 3A and FIG. 3B). Choosing local peaks from the absolute values of Sₕₑₐᵣₜ may not accurately segment the heart beats since the residual noise from the high-pass filter creates fake peaks; a more restrictive band-pass filter could reduce this noise but may also reduce temporal resolution.

Accordingly, a segmentation technique may be used in segmentation 212 that finds both the segmenting points and the rotation of each segment simultaneously. The shapes of consequent heartbeat arcs may be similar after accounting for temporal scaling due to different R-R intervals and a rotation between them due to residual breathing motion. The technique finds the segmenting point and the corresponding rotation transformation for each segment, where one segment post-rotation is most similar to its previous segment after scaling to be the same duration. The segmentation method may be non-iterative, may account for rotations, and may rely on comparison only between adjacent segments.

To measure the distance metric between segments sᵢ and sᵢ₊₁, their lengths may first be normalized to the longer segment using linear interpolation. The best rotation may then be computed by minimizing the mean square error between sᵢ and the rotated sᵢ₊₁. This rotation is given by,

$${s}_{i + 1}^{( r o t )} = {s}_{i + 1} \sqrt{\frac{{s}_{i} {s}_{i + 1}^{*}}{{s}_{i} + 1 {s}_{i}^{*}}} \qquad ( Equation 11 )$$

Given two complex vectors x and y with L elements each, the rotation angle, θ, that minimizes the mean square error:

$$E = {\sum }_{i = 1}^{L} ( {x}_{i} exp ( j θ ) - {y}_{i} ) {( {x}_{i} exp ( j θ ) - {y}_{i} )}^{*} = {\sum }_{i = 1}^{L} {x}_{i} {x}_{i}^{*} - {x}_{i} {y}_{i}^{*} exp ( j θ ) - {x}_{i}^{*} {y}_{i} exp ( - j θ ) {y}_{i} {y}_{i}^{*} \qquad ( Equation 12 )$$

This can be computed by setting the first derivative to 0, as follows:

$$\frac{dE}{d θ} = {\sum }_{i = 1}^{L} - {jx}_{i} {y}_{i}^{*} exp ( j θ ) + {jx}_{i}^{*} {y}_{i} exp ( - j θ ) = 0 \qquad ( Equation 13 )$$

Thus, an optimal rotation is given by,

$$exp ( j θ ) = \sqrt{\frac{{x}^{*} y}{{y}^{*} x}} \qquad ( Equation 14 )$$

The distance metric between two segments may then be defined as,

$$d ( {s}_{i} , {s}_{1 + 1} ) = \frac{{‖ {s}_{i} - {s}_{i + 1}^{( r o t )} ‖}_{2}^{2}}{{‖ {s}_{i} + {s}_{i + 1}^{( r o t )} ‖}_{2}^{2}} \qquad ( Equation 15 )$$

Once each heart beat segment is identified, its mid-point may be used as the timing for the corresponding heartbeat, which may then be used to compute the heart rate and R-R intervals.

In health metric calculation 214, using the segmented waveform, a variety of health metrics may be calculated. For example, the processor 118 may be used to calculate health metrics. The health metrics may include, instantaneous heart rate, average heart rate, and/or R-R intervals. The health metrics may be stored, displayed, and/or communicated to other computing systems.

In disease detection 216, a disease (e.g., condition) may be detected based on the health metrics. For example, the processor 118 may be used to detect a disease based on the health metrics. In other examples, other processors and/or computing systems may receive and/or generate the health metrics and perform the detection of disease. Examples of diseases which may be detected include heart rate variability, atrial fibrillation, and/or arrhythmias. Note that heart rate variability may be related to emotion, so emotional states may be detected in disease detection 216 based on heart rate variability in some examples. R-R intervals and/or heart rate variability calculated in accordance with examples described herein may be used, e.g., by processor 118 of FIG. 1, to distinguish between atrial fibrillation and sinus rhythm. It may also be used, e.g., by processor 118 of FIG. 1, to monitor stress, anxiety and the general health of the autonomic nervous system.

The detected disease may be stored, displayed and/or communicated to other computing systems. In some examples, other action may be taken based on the detected disease. For example, emergency responders may be contacted—e.g., the processor 118 of FIG. 1 may be used to initiate an emergency response message. In other examples, other actions may be taken. For example, if the system is deployed in an automobile and the subject is driving, the automobile may begin and/or stop autonomous operation responsive to detection of a disease. For example, if atrial fibrillation is detected, the automobile may initiate autonomous operation for the safety of the driver and other drivers. In another example, if the heart rate decreases to a level that the subject is suspected to be asleep, an autonomous vehicle may take action to wake the driver—including vibrating all or portions of the car and/or emitting loud noises and/or stopping the vehicle.

Generally, the heart beat detection techniques described herein may be utilized for time-to-time spot monitoring of one or more subjects. For example, an electronic device (e.g., the smart speaker 104 of FIG. 1) may receive an indication to start monitoring and/or may start monitoring at a particular time or interval. In some examples, the indication to start monitoring may be received through a user interface, such as user interface 128 of FIG. 1. Responsive to the indication to start monitoring, the electronic device may generate and provide one or more interrogation signals as described herein, calculating beamforming weights, and generate beamformed waveforms indicative of heart beats of the subject. During a monitoring period, the subject may be requested to remain still (e.g., not engage in large gross motions which may obscure the signal). In other examples, continuous monitoring of an environment may be made. During times when continuous monitoring results in beamformed waveforms that are not indicative of heart beats, the portion of the waveform may be discarded. In some examples, portions of reflected signals and/or beamformed waveforms occurring during large motion of the subject may be discarded and not utilized to detect heart beats and/or health metrics. In some examples, systems described herein may detect motion of a subject greater than a threshold (e.g., using the interrogation signals), and may stop or pause cardiac monitoring responsive to the detection of large motion of the subject. When the subject has stopped significant motion, cardiac monitoring as described herein may resume, including calculation of an updated set of beamforming weights in some examples.

FIG. 3A depicts, for a healthy subject, an ECG trace and a corresponding waveform generated in accordance with examples described herein. The x-axis of the graphs in FIG. 3A is time (in seconds). The ECG trace 302 may be representative of a measurement taken by a conventional ECG of a healthy subject. For example, the ECG trace 302 may be an ECG measurement for the subject 102 of FIG. 1 and/or the subject of FIG. 2. The ECG trace 302 has a noticeable peak at each heartbeat. The heart rhythm indicated by ECG trace 302 is normal (e.g., it is relatively regular with characteristically-shaped peaks).

FIG. 3A depicts a corresponding waveform 304 which may be generated by example systems described herein, for the same healthy subject as the ECG trace 302. The waveform 304 may be, for example, generated by the processor 118 of FIG. 1 as a result of beamforming received signals from the microphones of FIG. 1 based on the beamforming weights 130. The waveform 304 may represent an output of the beamforming 210 operation of FIG. 2. Note that, because the beamforming weights may be complex, the waveform 304 has both an in-phase component 306 and a quadrature component 308. The cardiac rhythm signal may shift between the in-phase and quadrature phase components due in part to residual respiration motion that may remain even after beamforming.

The dotted vertical lines in FIG. 3A represent the divisions between segments. Each segment generally corresponds with a heartbeat, as can be seen by comparing the waveform 304 with the ECG trace 302. The segments may be identified by, for example, processor 118 of FIG. 1 using a segmentation operation. The segments may be identified by segmentation 212 of FIG. 2.

FIG. 3B depicts, for a subject experiencing atrial fibrillation, an ECG trace and a corresponding waveform generated in accordance with examples described herein. The x-axis of the graphs in FIG. 3B is time (in seconds). The ECG trace 312 may be representative of a measurement taken by a conventional ECG of a subject experiencing atrial fibrillation. For example, the ECG trace 312 may be an ECG measurement for the subject 102 of FIG. 1 and/or the subject of FIG. 2. The ECG trace 312 has a noticeable peak at each heartbeat. The heart rhythm indicated by ECG trace 312 is abnormal and indicative of atrial fibrillation (e.g., it has irregular peaks and an absence of P-waves).

FIG. 3B depicts a corresponding waveform 314 which may be generated by example systems described herein, for the same subject experiencing atrial fibrillation as the ECG trace 312. The waveform 314 may be, for example, generated by the processor 118 of FIG. 1 as a result of beamforming received signals from the microphones of FIG. 1 based on the beamforming weights 130. The waveform 314 may represent an output of the beamforming 210 operation of FIG. 2. Note that, because the beamforming weights may be complex, the waveform 314 has both an in-phase component 316 and a quadrature component 318. The cardiac rhythm signal may shift between the in-phase and quadrature phase components due in part to residual respiration motion that may remain even after beamforming.

The dotted vertical lines in FIG. 3B represent the divisions between segments. Each segment generally corresponds with a heartbeat, as can be seen by comparing the waveform 314 with the ECG trace 312. The segments may be identified by, for example, processor 118 of FIG. 1 using a segmentation operation. The segments may be identified by segmentation 212 of FIG. 2.

Note that, in traditional ECG traces, the trace is a plot of voltage picked up by the ECG sensor(s). Accordingly, utilizing ECG traces, heart rhythms are typically analyzed based on voltage. However, beamformed waveforms used to detect heart rhythms described herein are instead representative of motion of a subject caused by cardiac function. Accordingly, examples of systems and methods described herein detect heart rhythms, calculate health metrics, and/or detect disease based on motion of a subject induced by cardiac activity. This is in contrast to systems utilizing voltage to detect heart rhythms.

### IMPLEMENTED EXAMPLES

A clinical study was conducted with both healthy participants and hospitalized cardiac patients with diverse structural and arrhythmic cardiac abnormalities including atrial fibrillation, flutter and congestive heart failure. Compared to electrocardiogram (ECG) data, the example system used computed R-R intervals for healthy participants with a median error of 28 ms over 12,280 heart beats and a correlation coefficient of 0.929. For hospitalized cardiac patients, the median error was 30 ms over 5,639 heart beats with a correlation coefficient of 0.901. The increasing adoption of smart speakers in hospitals and homes may provide a means to realize the potential of example non-contact cardiac rhythm monitoring systems described herein for monitoring of contagious or quarantined patients, skin sensitive patients and in telemedicine settings.

A cohort of 26 voluntary participants were recruited who had no prior history of cardiac conditions. The median age of the participants was 31 [interquartile range (IQR), 8.5] years and body mass index (BMI) was 22 (IQR, 3). The female-to-male ratio was 0.6. Cardiac patients were enrolled prospectively from the acute care general cardiology unit at the University of Washington Medical Center, a tertiary academic medical center in an urban area. All patients' heart rates and rhythms were continuously monitored in this unit using hospital-commissioned, three-lead surface electrode telemetric monitoring systems.

Patients were eligible for inclusion if they were older than 18 years of age and able to provide informed consent. They were excluded if they were unable to sit still for more than 15 minutes, demonstrated cardiopulmonary instability, or had altered mental status as determined by a medical doctor (D.N.). Randomization was not applicable, and study investigators were not blinded. Once enrolled in the study, patients had their clinical variables—age, gender, height, weight, BMI, medications, and medical comorbidities—abstracted from their electronic medical records This study was approved by the University of Washington Institutional Review Board.

In the study, the EliteHRV Corsense PPG and Polar H10 ECG sensors were used for ground truth. PPG sensors are known to produce comparable R-R interval accuracies to ECG, with high correlation coefficients between 0.968 and 0.998. To verify this, a comparison test was performed between the ground truth sensors on two healthy participants and noted that the mean absolute R-R interval difference was 11 ms.

Participants were fitted with a Polar H10 Sensor System (Polar Electro, Kempele, Finland) that measures ECG and outputs the heart rate and R-R intervals. The ECG sensor was used to gather ground truth data for the study. All testing was performed in a private room at University of Washington, where participants sat upright on a chair by a table on which an example smart speaker, such as one in accordance with the schematic shown and described with reference to FIG. 1, was placed.

The testing was conducted with the clothing the participants were already wearing indoors such as blouses, tops, T-shirts, and button downs made with different fabric materials. Participants took a series of one-minute measurement sessions, where they were asked to sit still and breathe normally.

For each healthy participant, a total of seven 60-second sessions were conducted. In the first three, the smart speaker was placed in front of the participant's chest at the nipple level, at a distance of 40 cm, 50 cm and 60 cm. For the fourth session, the smart speaker was pointed 10 cm above the participant's chest at a distance of 50 cm. For the fifth, the smart speaker was pointed towards the chest but at an angle of 20° and a distance of 50 cm. In the sixth, measurements were conducted at a distance of 50 cm, while jazz music played at around 75 dB (A) sound power level from a distance of 5 m. In the final session, participants were asked to jog in place to increase their heart rate above 110 beats per minute (BPM) before starting measurements at a distance of 50 cm. Note that these distances and angles and background noises are exemplary only, and in other examples other distances, angles, and/or background noises may be present.

Average heart rate was computed by counting the number of heart beats over a period of 60 seconds and it was compared to the heart rate output by the ECG device. Measurements from the smart speaker and the ECG sensor had intra-class and concordance correlation coefficients of both 0.983. The R-R intervals output by the smart speaker and the ECG sensor were also compared. The intra-class correlation coefficient (ICC) and concordance correlation coefficient (CCC) between the two measurements were 0.929 and 0.927 respectively.

Example system performance was also tested for hospitalized cardiac patients (n=24). Once enrolled in the study, the patients' existing telemetries were reviewed by a medical doctor, and the patients were adjudicated into either a regular rhythm category (e.g., sinus rhythm, atrial flutter with regular conduction, ventricular paced, or atrioventricular paced) or an irregular rhythm category (atrial fibrillation or atrial flutter with variable conduction). Patients in the irregular rhythm cohort were more likely to have a history of atrial fibrillation and more likely to be female. Age, BMI, reason for hospitalization, medical comorbidities, and cardiac medications were uniform between the regular and irregular rhythm cohorts. Since prior audiocardiography work showing poor results in extreme obese patients, patients whose BMI exceeded 35 were excluded for this study.

To obtain ground truth heart rate and R-R interval data for comparison, half the patients were fitted with a chest-worn Polar H10 Sensor System (Polar Electro, Kempele, Finland). Patients unable to wear the chest band due to discomfort, recent thoracic surgery, or poor ECG signal acquisition (n=12) were fitted with a fingertip-worn CorSense monitor (Elite HRV, Asheville, North Carolina, USA). These data were downloaded in real time to a Bluetooth-connected smartphone using the HRV+ mobile app (Elite HRV, Asheville, North Carolina, USA). The rationale behind this method is that hospital telemetry software does not allow for digitalization and storage of the R-R interval data. Previous studies have demonstrated portable heart rate variability (HRV) devices to have acceptable error compared to gold standard ECG monitoring.

Patients were positioned sitting vertically on the hospital beds in their own room and the smart speaker system, such as that shown and described with respect to FIG. 1, was placed around 50 to 60 cm from them, with the speaker inlet pointed at the chest at the level of the nipple. Ambient noise sources (e.g., television) were turned off and family members and visitors of patients who were required to stay in the room were asked to sit at least 2 meters away from the smart speaker during the sessions. Data was acquired from the smart speaker system in five sessions, each lasting 60 seconds. During each session, patient were instructed to remain still. All patients tolerated the data acquisition process; however, data acquisition was prematurely terminated for one patient due to developing nausea related to a prior medical condition.

The median absolute error in the heart rate calculated using the smart speaker system relative to ground truth collected through ECG data was 2 beats per minute, with a 90^{th} percentile error of less than 3 beats per minute. For R-R intervals, the intra-class correlation coefficient (ICC) and concordance correlation coefficient (CCC) were 0.901 and 0.898, respectively. The median absolute error in the R-R intervals was around 30 ms, with a standard deviation of 67.2 ms, and the 90^{th} percentile error was less than 93 ms. The mean absolute error in the R-R intervals as a percentage of the ground truth R-R interval was 4.0% with a standard deviation of 7.6%.

Focusing on irregular heartbeats, the mean absolute R-R interval error among patients with atrial fibrillation instances was 35 ms with intra-class correlation (ICC) and concordance correlation coefficients (CCC) of 0.891 and 0.890, respectively. Higher median R-R intervals correspond to higher 90-percentile error. There was no noticeable decrease in accuracy among those with irregular rhythms compared to those with regular rhythms. Within the context of clinical practice, it is unlikely that this magnitude of error would result in diagnostic errors for detecting atrial fibrillation where R-R interval variation less than 50 ms is often not clinically important. In atrial fibrillation, the R-R interval widely varies from beat to beat and standard deviations range between 95-233 ms in different physiological states. Proper diagnosis of rhythm disorders often relies on the ability to detect temporally disparate R-R intervals, rather than precise R-R interval measurement.

Data was collected from patients in the cardiac floor of a tertiary care medical center with a variety of cardiac conditions, which included cardiac conduction disorders, arrhythmias, cardiomyopathy as well as valvular disorders. Many of these cardiac conditions directly or indirectly affect the heart rate variability. Respiratory sinus arrhythmia, which is a major cause of heart rate variability becomes less common with age and is less prevalent in patients with diabetes due to autonomic neuropathy. The hospitalized population had a mean age of 63.2 years in the regular rhythm group and 68.0 in the irregular rhythm group, and there were a total of 5 out of 24 patients with diabetes. In addition, medications that influence vagal tone, such as beta blockers, digoxin, opiate pain medications may decrease sinus arrhythmia. The sample of hospitalized cardiac patients often had multiple factors which could reduce heart rate variability.

The smart speaker used in the study included a seven-microphone array, which had an identical microphone layout and sensitivity to the Amazon Echo Dot, but had an ability to output raw recorded signals. The prototype included a commercial UMA-8-SP USB circular array with 7 microphones with a 4.3 cm separation, similar to an Amazon Echo Dot; a PUI Audio AS05308AS-R speaker; and a 3D-printed case that held the microphone array and the speaker next to each other. The smart speaker was connected to a computer via USB as an external sound card device, where sounds were played and recorded at a sampling rate of 48 kHz and a sound pressure level of around 75 dB at a distance of 50 cm.

The signal processing of FIG. 2 was used to obtain heart beats and/or health metrics for study participants.

From the foregoing it will be appreciated that, although specific embodiments have been described herein for purposes of illustration, various modifications may be made while remaining with the scope of the claimed technology.

Examples described herein may refer to various components as “coupled” or signals as being “provided to” or “received from” certain components. It is to be understood that in some examples the components are directly coupled one to another, while in other examples the components are coupled with intervening components disposed between them. Similarly, signals may be provided directly to and/or received directly from the recited components without intervening components, but also may be provided to and/or received from the certain components through intervening components.

What is claimed is:

1. A method comprising:
provide an interrogation signal from a speaker;
receive at least one reflection of the interrogation signal from a subject, the at least one reflection received at multiple microphones to provide multiple received signals;
generating a waveform indicative of heart beats of the subject at least in part by beamforming the multiple received signals.

2. The method of claim 1, wherein the interrogation signal comprises a signal in an inaudible range.

3. The method of claim 1, wherein the interrogation signal comprises a frequency modulated continuous wave (FMCW) signal having a frequency between 18 and 22 kHz.

4. The method of claim 1, wherein the interrogation signal comprises white noise.

5. The method of claim 1, wherein the subject comprises a human body.

6. The method of claim 1, wherein said beamforming the multiple received signals comprises weighting the multiple received signals in accordance with beamforming weights.

7. The method of claim 6, further comprising calculating the beamforming weights at least in part by evaluating a function configured to increase a portion of the waveform corresponding to the heart beats and decrease a portion of the waveform corresponding to breathing motion of the subject.

8. The method of claim 7, wherein evaluating the function comprises performing a gradient ascent technique.

9. The method of claim 7, wherein the function is based in part on expected frequencies of respiration and the heart beats.

10. The method of claim 6, wherein the beamforming weights are complex weights.

11. The method of claim 1, further comprising segmenting the waveform into segments corresponding to heart beats of the subject.

12. The method of claim 1, further comprising calculating a heart rate, an inter-beat interval, or combinations thereof based on the waveform.

13. The method of claim 1, further comprising calculating an inter-beat interval based on the waveform and detecting atrial fibrillation, flutter, congestive heart failure, arrhythmia, or combinations thereof based at least in part on the inter-beat interval.

14. An apparatus comprising:
a speaker;
a signal generator coupled to the speaker, the signal generator configured to drive the speaker to provide an interrogation signal;
a plurality of microphones, the plurality of microphones configured to receive at least one reflection of the interrogation signal from a subject and provide reflected signals;
at least one processor configured to beamform the reflected signals to generate a waveform indicative of heart beats of the subject.

15. The apparatus of claim 14, wherein the signal generator is configured to drive the speaker to provide the interrogation signal comprising a frequency modulated continuous wave (FMCW) signal having a frequency between 18 and 22 kHz.

16. The apparatus of claim 14, wherein the at least one processor is configured to beamform the reflected signals by weighting the reflected signals in accordance with beamforming weights.

17. The apparatus of claim 16, wherein the at least one processor is configured to calculate the beamforming weights at least in part by evaluating a function configured to increase a portion of the waveform corresponding to the heart beats and decrease a portion of the waveform corresponding to breathing motion of the subject.

18. The apparatus of claim 17, wherein the function is based in part on expected frequencies of respiration and the heart beats.

19. The apparatus of claim 14, wherein the at least one processor is further configured to calculate a heart rate, an inter-beat interval, or combinations thereof based on the waveform.

20. The apparatus of claim 19 further comprising a communication interface coupled to the at least one processor, the communication interface configured to transmit the waveform, the heart rate, the inter-beat interval, or combinations thereof to another computing device.
